(19) United States
(12) Patent Application Publication
(10) Pub. No.: US 2021/0290107 A1
(43) Pub. Date: Sep. 23, 2021

(54) JOINT MOBILITY MEASUREMENT DEVICE

(71) Applicant: William Thomas Murray, West Chester, PA (US)

(72) Inventor: William Thomas Murray, West Chester, PA (US)

(21) Appl. No.: 16/610,940

(22) PCT Filed: May 14, 2018
(86) PCT No.: PCT/US2018/032608

§ 371 Date: Nov. 5, 2019

Related U.S. Application Data
(60) Provisional application No. 62/505,854, filed on May 13, 2017.

(51) Int. Cl. A61B 5/11; A61B 5/00

(52) U.S. Cl. CPC A61B 5/1121; A61B 5/1126; A61B 5/0002; H04W 4/80; A61B 5/6831; A61B 5/6895; A61B 5/742

(57) ABSTRACT

A device to measure the range of motion of any joint with accuracy without having to place the device exactly on the joint to be measured. The device comprises of an internal measurement unit which measures the change in motion and send the information to the microprocessor which processes the information to accurately show the range of motion to within a degree, and the speed and acceleration with which the movement of the joint occurred. The microprocessor then sends the range of motion, speed and acceleration to an external device to be displayed. The external device then displays a 2-D or 3-D rendering of the movement.

### CROSS-REFERENCE

This application claims priority to U.S. Provisional Application Ser. No. 62/505,854 filed on May 13, 2017.

### BACKGROUND OF THE INVENTION

This is a device that can be used to measure joint mobility. In order to help build muscle or to measure the mobility of a joint, the currently available methods either involve tedious manual measurements or bulky devices that are not very accurate. For example, there are devices that are used to measure joint mobility after an orthopedic surgery are bulky, not very accurate and sometimes hard to read, but mostly, the medical joint mobility measurement devices require that the user estimate where the joint is which often is the cause for inaccuracies. Additionally, a number of exercises involving joints are manually judged. For example, squats. The current way squats are judged for competition is as follows, Three judges watch a lifter. The three judges are positioned around the lifter such that there is one judge on each side and one judge in the front. This leaves room for human error.

Thus, there is a need for a joint mobility measurement device that reduces inaccuracies, does not need estimation about its placement, and is versatile enough to be used on any joint.

### SUMMARY OF THE INVENTION

The present invention is related to a range of motion measurement device to accurately digitally measure the angular movement of any joint and display the angle, speed and acceleration, of the movement on an external device.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1, is the top view of the range of motion measurement device without its outer cover containing the core components of the device.

FIG. 2, is the bottom view of the range of motion device without its outer cover.

FIG. 3, is a diagram of the device's rotation coordinate system for Euler Angles.

FIG. 4, is an embodiment of how the device can be used to measure the range of motion of the shoulder joint.

FIG. 5, is an embodiment of how the device can be used to measure the range of motion of the hip joint.

FIG. 6 is an illustration of a user performing a squat with the device on their thigh and angles of measurement shown.

FIG. 7 is an illustration of a user performing a squat with the device on the bar.

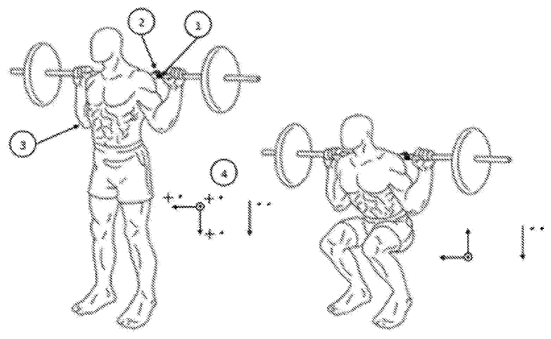

FIG. 8 is an illustration of a user performing a bench press with the device on the bar.

FIG. 9 is an illustration of an exemplary embodiment of how knee flexion using a standard goniometer can be measured.

FIG. 10 is an illustration of a hip abduction using a standard goniometer.

FIG. 11 is an illustration of a hip rotation around the axis of the leg.

FIG. 12 is an illustration of a side lateral raise where the user is standing with a dumbbell in each hand with the device on, the dumbbell; the user performs a shoulder lateral raise movement raising their arms in a pitch/roll motion according to the coordinate system.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

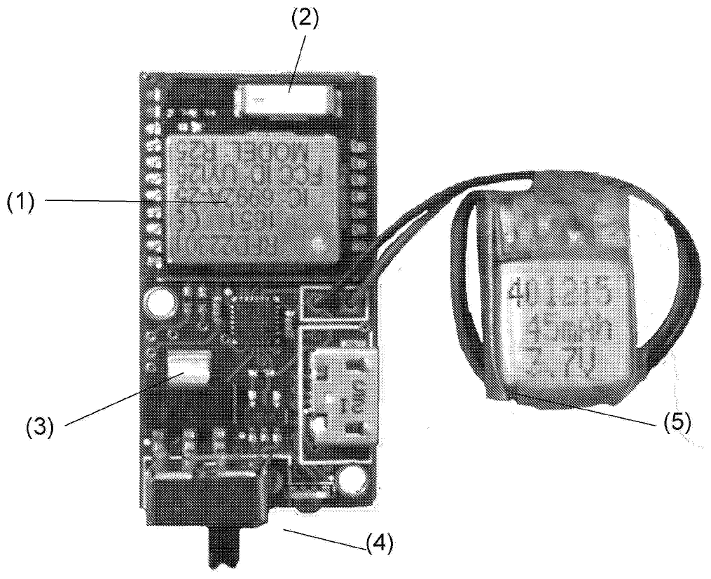

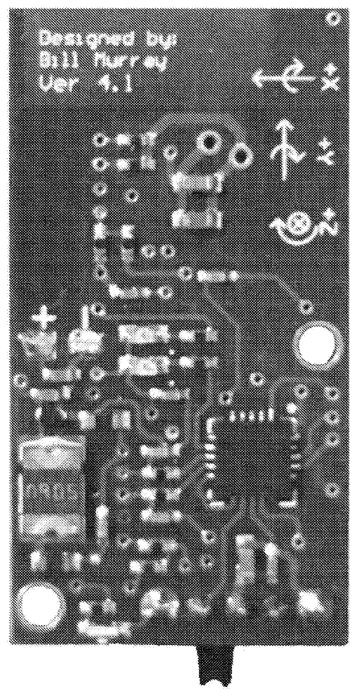

FIGS. 1 and 2 depict a digital joint mobility measurement device. The device comprises of a microcontroller and Bluetooth integrated circuit (1), an antenna (2), a 9-axis internal measurement unit (IMU) (3), a USB port (4), and a battery (5). The microcontroller reads and processes the input from the IMU and sends the relevant information to an external device via Bluetooth and the antennae.

In some embodiments, there is one antenna (2). In some other embodiments, there are more than one antennae. In some embodiments, there are two antennae.

In some embodiments, the microcontroller may have one USB port (4). In some other embodiments, the microcontroller may have more than one USB ports. In some embodiments, the microcontroller may have 2 USB ports.

In some embodiments, the USB port(s) may be for the type USB 2.0. In some embodiments, the USB port(s) may be for the type USB 3.0. In some embodiments, the USB ports may be a combination of any types of USB. In some embodiments, the USB port(s) may be for the type Micro-USB A. In some embodiments, the USB port(s) may be for the type Micro USB B. In some embodiments, the USB port(s) may be for the type USB B-type. In some embodiments, the USB port(s) may be for the type USB C-type. In some embodiments, the USB port(s) may be for the type USB A-type.

In some embodiments, the microcontroller (1) is programmed using the USB port (4). In some other embodiments, the microcontroller is pre-programmed.

The microcontroller (1) receives the information from the IMU (2) and processes it. In some embodiments, the microcontroller (1) sends out the processed information to the external device where it is displayed. In some preferred embodiments, the microcontroller (1) then sends the processed results out to the external device where it is displayed in a mobile application.

In some embodiments, the microcontroller (1) is powered by the battery (5). In some embodiments, the microcontroller is from the charge by the USB port.

In some embodiments, the IMU (2) comprises of a gyroscope, accelerometer, and/or a magnetometer. In some embodiments, the IMU comprises of a 3-Axis gyroscope, a 3-Axis accelerometer, and/or a 3-Axis magnetometer.

In some embodiments, the IMU communicates with the microcontroller via a synchronous, multi-master, multi-slave, packet switched, single-ended, serial computer bus. In some embodiments, the bus may be an asynchronous, multi-master, multi-slave, packet switched, single-ended, serial computer bus. In some embodiments, the bus may be a synchronous, multi-master, multi-slave, packet switched, serial computer bus. In some embodiments, the bus may be an asynchronous, multi-master, multi-slave, packet switched, serial computer bus. In some embodiments, the bus may be a synchronous, multi-master, multi-slave, packet switched, parallel computer bus. In some embodiments, the bus may be an asynchronous, multi-master, multi-slave, packet switched, parallel computer bus.

The USB port (4), is used to upload firmware to the microcontroller to program the microcontroller from an external device. The USB port may also be used to charge the battery (5). The USB port may also be used to communicate with external devices.

The battery (5) may be a rechargeable or non-rechargeable battery. In some embodiments, the battery could be a lithium-ion battery. In some embodiments, it can be a lithium-polymer battery. In some embodiments, it can be a nickel-cadmium battery. In some embodiments, it can be a nickel-metal hydride battery. In some embodiments, the device also can measures and track the battery percentage. This information is also sent to the external device and/or associated software or application. The device can use a voltage divider (e.g., with 2 resistors) and can measure the voltage using an analog input on the microcontroller.

In some embodiments the device uses quaternions to measure the range of motion. In some embodiments, the device uses Euler angles to measure the range of motion.

The microcontroller receives quaternions provided by the MU of the following form:

\vec{q}=w+xi+yj+zk w,x,y,x∈ [symbol] (1)

where i²=j²=k²=−1. The microcontroller unit will then transmit these quaternions, R=[w, x, y, z], to the external device via Bluetooth communication via the on-board antenna. The microcontroller will stop transmitting information to the external device when the microcontroller receives a separate signal from the external device for this function. When transmitting, the external device will record the first set of quaternions received, \vec{q}₀, as the initial value and position. This is the initial position of the user's body part and joint rotation. From here the user will move their body part through a specified movement and each quaternion that is received after the initial value, \vec{q}ᵢ, will be compared to the initial quaternion using a four-dimensional (4D) dot-product, shown below in equation (2):

$$cos ( \frac{θ}{2} ) = \frac{{\vec{q}}_{0} \cdot {\vec{q}}_{i}}{‖ {\vec{q}}_{0} ‖ ‖ {\vec{q}}_{i} ‖} \qquad ( 2 )$$

where θ is the total angle traveled from the initial position in radians. The angle is then converted to degrees. The data is recorded as sets and repetitions for multiple measurements. The data is also plotted and saved as two- and three-dimensional plots. The two-dimensional plot will show angle vs repetitions and is selectable by sets Not only is the angle of the joint recorded, but a three-dimensional plot of the device's motion through space is also recorded by the following method.

$${\vec{v}}_{n} = [ \begin{matrix}{v}_{{n}_{1}} \\ {v}_{{n}_{2}} \\ {v}_{{n}_{3}}\end{matrix} ] = R {\vec{v}}_{i n} = [ \begin{matrix}{a}_{11} {a}_{12} {a}_{13} \\ {a}_{21} {a}_{22} {a}_{23} \\ {a}_{31} {a}_{32} {a}_{33}\end{matrix} ] [ \begin{matrix}{v}_{i {n}_{1}} \\ {v}_{i {n}_{2}} \\ {v}_{i {n}_{3}}\end{matrix} ] \qquad ( 3 )$$

Equation 3 describes the rotation of the device through space, \vec{v}ₙ, where \vec{v}ₙ is a constant vector in Cartesian space. \vec{v}ᵢₙ is also a constant vector in Cartesian space and R is a rotation matrix.

$${\vec{v}}_{in} = [ \begin{matrix}\frac{1}{\sqrt{3}} \\ \frac{1}{\sqrt{3}} \\ \frac{1}{\sqrt{3}}\end{matrix} ] \qquad ( 4 )$$

Equation 4 shows \vec{v}ᵢₙ, which is chosen such that the points in space always lie on a unit sphere.

$$R = [ \begin{matrix}{a}_{11} {a}_{12} {a}_{13} \\ {a}_{21} {a}_{22} {a}_{23} \\ {a}_{31} {a}_{32} {a}_{33}\end{matrix} ] = \\ [ \begin{matrix}1 - 2 {q}_{2}^{2} - 2 {q}_{3}^{2} & 2 ( {q}_{1} {q}_{2} - {q}_{0} {q}_{3} ) & 2 ( {q}_{1} {q}_{3} - {q}_{0} {q}_{2} ) \\ 2 ( {q}_{1} {q}_{2} - {q}_{0} {q}_{3} ) & 1 - 2 {q}_{1}^{2} - 2 {q}_{3}^{2} & 2 ( {q}_{2} {q}_{3} - {q}_{0} {q}_{1} ) \\ 2 ( {q}_{1} {q}_{3} - {q}_{0} {q}_{2} ) & 2 ( {q}_{2} {q}_{3} - {q}_{0} {q}_{1} ) & 1 - 2 {q}_{1}^{2} - 2 {q}_{2}^{2}\end{matrix} ] = Rotation Matrix \qquad ( 5 )$$

Equation 5 shows the rotation matrix, R, which is obtained from each quaternion, \vec{q}ᵢ, that is received from the device. Each value of \vec{v}ₙ will correspond to a point in space on a unit sphere corresponding to the incoming quaternion.

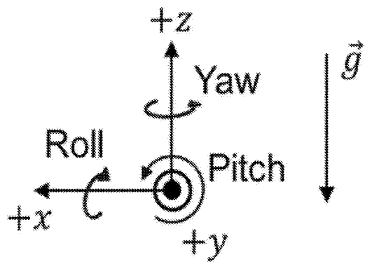

The second method to measure range of motion uses Euler angles provided by the IMU as yaw, pitch, and roll, which are sent to the microcontroller. FIG. 3 shows the coordinate system of the device with respect to gravity regardless of device orientation. The coordinate system of the device's rotation will always be oriented such that yaw moves in the xy-plane regardless of how the device is oriented. Pitch is described as rotation in the xz-plane, roll is described as rotation in the yz-plane, and yaw is described as rotation in the xy-plane as shown in FIG. 3. The angular change of any device rotation in the pitch or roll planes can be calculated as the summation of pitch and roll angles regardless of the device orientation with respect to the coordinate system in FIG. 3.

The device also calculates velocity and acceleration using the data from the IMU. The raw acceleration values read from the IMU are inherently noisy. This is a well-documented problem. To de-noise the acceleration values, we implement a high pass filter using a weighted running average. Consider the following raw acceleration values read from the IMU from time samples 1 through T.

a_{raw}(t)=a_{raw}(1)a_{row}(2) . . . a_{raw}(T) (1)

To smooth and de-noise the data, we implement a running weighted average over the previous M samples with a weighting, factor of β. Adjusting the weighting factor adjusts how much smoothing is applied to the sampling. An especially low value for β will eliminate noise spikes but will also over smooth the data while a high weighting factor will not effectively eliminate noise spikes. After filtering, the acceleration data is as follows. These filtered acceleration values calculated separately for the x, y, and z directions.

$${a}_{filtered} ( T ) = \frac{{( {{a}_{raw} ( T )}^{β} + {{a}_{raw} ( T - 1 )}^{β} … + {{a}_{raw} ( T - M )}^{β} )}^{\hat{}} ( 1 / β )}{M} \qquad ( 2 )$$

The filtered acceleration values are used to calculate the velocity along x, y, and z. The velocity along one axis at a time T is calculated by:

v(T)=∫₀^{T}a_{filtered}(t)dt (3)

When taking the integral, there is an unknown initial condition which we set as zero since we assume the device is not in motion at the beginning of data acquisition. In calculating velocity data from acceleration data, there is a well-documented problem of linear drift in the velocity. To compensate for this, we implement a first order polynomial fit to the velocity data which is subtracted off using the constraints the device is stationary at the beginning and end of data acquisition.

$$\begin{matrix}{v}_{c o r r e c ted} ( t ) = v ( t ) - p ( t ) & ( 4 ) \\ p ( t ) = \frac{( v ( T ) - v ( 0 ) )}{T} t + v ( 0 ) & ( 5 )\end{matrix}$$

Because the corrected velocity depends on the initial and final condition of the devices, it must be calculated after data acquisition by the IMU has ended. The above calculation provides the corrected velocity values along the x, y, and z axes. Furthermore, we calculated the overall speed of the device by taking the magnitude of the velocity.

$$s ( t ) = \sqrt{{{v}_{{x}_{corrected}} ( t )}^{2} + {{v}_{{y}_{corrected}} ( t )}^{2} + {{v}_{{z}_{corrected}} ( t )}^{2}} \qquad ( 6 )$$

The device in FIG. 1 can be enclosed in a case. The case may be made of any light and rigid material in some embodiments, the case may be made of a plastic polymer. In some embodiments, the case may be made of metal. In some embodiments, the case may be made of aluminum. In some embodiments, the case may be made of steel. In some embodiments, the case may be made of stainless steel.

The device enclosed in a case is then placed on the body of the individual whose range of motion is to be measured. The case may be temporarily affixed to the body of the individual using an elastic band. In some embodiments, the case may be temporarily affixed to the body of the individual using a velcro strapping.

To measure the range of motion of a joint, the device can be placed as far away from the joint to measure and where the body part is moving, but before the next closest joint (e.g. if you were measuring the elbow, you would place the device on the wrist, not the bleep). Once the device is placed farthest away from the joint to measure but before the next joint, the device is turned on while keeping the patient stationary. After the device is on, the patient may be asked to move the body part on which the device is placed to measure the range of movement accurately from the stationary position, Once the movement is performed, the device sends information to the microcontroller, which then processes the data based on the gyroscope, accelerometer and/or magnetometer readings, to give an accurate range of motion readings. In one non-limiting example, to measure range of motion, the device can be attached as far away on the body part that will move throughout the range of motion measurement connected to the joint in question, but before the next closest joint. This reduces any false or noisy data from other joint movement.

The device that is disclosed in this invention can be used to measure the range of motion of any joint. In some embodiments it may be used to measure the range of motion of a hinge joint. In some other embodiments, it may be used to measure the range of motion of a pivot joint. In some other embodiments, it may be used to measure the range of motion of a ball and socket joint. In some other embodiments, it may be used to measure the range of motion of a saddle joint. In some other embodiments, it may be used to measure the range of motion of a gliding joint. In some other embodiments, it may be used to measure the range of motion of a planar joint.

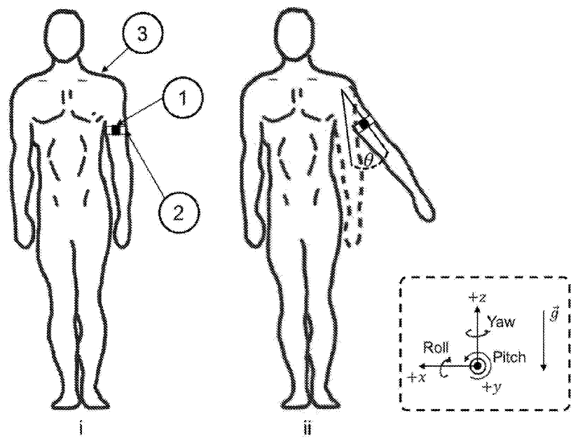

FIG. 4, is an embodiment of how the device can be used to measure the range of motion of a ball and socket joint. In FIG. 4, the device is used to measure the range of motion of a patient's shoulder. The range of motion here is the angle between where the arm is positioned when starting the movement, and where the arm is any time after that recorded first value. The device is placed as far away from the shoulder joint as possible but before the next closest joint, i.e., the elbow. After placing and securing the device in its location on the patient, the device is turned on while the patient is still. The patient then moves her hand in different directions per the request of the physician. The IMU measures the movement in different directions during regular time intervals and sends the information to the microcontroller. The microcontroller then processes the information and sends the output in radians and plot points to the external device.

The external device may plot this information as a two-dimensional or three-dimensional map, showing the patient's motion through space.

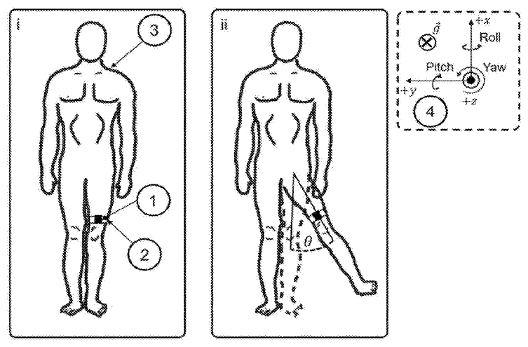

FIG. 5 is another embodiment of how the device can be used to measure the range of motion of the hip joint. The device is placed on the patient's thigh just above the knee but as far away from the hip joint as possible. The range of motion here is the The patient then moves her legs in different directions per the request of the physician. The IMU measures the movement during regular time intervals and sends the information to the microcontroller. The microcontroller then processes the information and sends the output in radians and plot points to the external device.

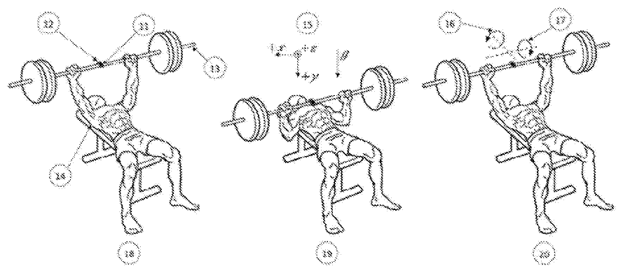

FIG. 8, depicts a user performing a bench press with the device strapped to the bar. The current way the bench press is judged for competition is as follows Three judges watch a lifter. The three judges are positioned around the lifter such that there is one judge on each side and one judge in the front. This leaves room for human error. FIG. 18 shows a bench measurement device 11, which includes electronics and outer enclosure. The bench measurement device 11 is attached to a band 12 that wraps around the barbell 13. FIG. 18 shows a 3-dimensional coordinate system 15 that refers to the internal motion unit (IMU) of the bench measurement device 11 as well as the force vector gravity for reference.

The bench measurement device notifies the user using mobile application software when the user meets the user's 14 bench goal based on movement through the coordinate system 15 with respect to gravity and the rotation of the barbell 16 and 17, as well as calibration settings used in the mobile application software. The mobile application software is usually stored in a mobile device, such as a phone. The bench measurement device includes a sensor. The circuitry of the sensor and sensor parts are shown in FIG. 8, numbers 19-26. The sensor is a small electronic device that includes a microcontroller, accelerometer, gyroscope, Bluetooth chip, antenna, USB chip, and micro-USB port enclosed by a case. The case attaches to the barbell via an elastic band and is used to determine the start 18, press 19 and rack 20 commands of the users bench press, as well as the pitch 15 and yaw 16 of the barbell 13.

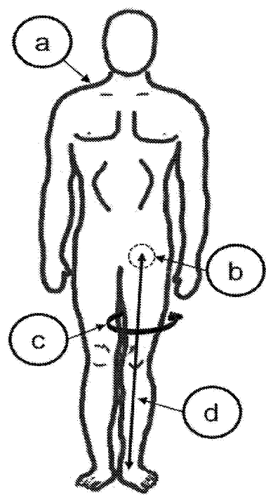

The case attaches to the barbell via an elastic band and is used to determine the pitch and yaw of the barbell, the powerlifting competition commands of start, press and rack, and the velocity and acceleration of the bench press. The sensor when attached to the user reads and analyzes data to notify the user in real time if the barbell has any pitch or yaw and if the start, press and rack commands are achieved. The sensor communicates with the mobile application software via Bluetooth to the mobile device. Mobile application software on the mobile device or a computer was developed to notify the user if the start, press and rack commands are achieved, the pitch and yaw of the barbell, the velocity and acceleration of each bench press repetition, as well as calibration of these settings. The mobile application software stores this information within a profile the user will create. The definition of an acceptable bench press in the sport of powerlifting is as follows. The lifter unracks their weight and waits for the press command, which is when the barbell is completely motionless and the lifter is in position 18 in FIG. 11. Once the lifter receives the start command, the lifter brings the barbell down to their chest where the bar must touch their chest and become completely motionless as shown in position 19 in FIG. 11. Once the barbell is touching the lifters chest and the barbell is motionless, the lifter receives the press command Once the press command is received, the lifter will press the barbell to position 20 in FIG. 11. Once the lifter is motionless in position 20, the lifter receives the rack command where the lifter proceeds to place their weight back in the rack. The majority of weight lifters do not practice the sport of powerlifting, but the practice of having, no pitch or yaw of the barbell throughout the bench as well as full range of motion as shown in FIG. 11, 18-20 results in engaging all of the important muscles that are noted in a bench press. The sensor is intended for weight lifters of all skill levels—beginner, intermediate, advanced and professional.

While different embodiments of the invention have been described in detail herein, it will be appreciated by those skilled in the art that various modifications and alternatives to the embodiments could be developed in light of the overall teachings of the disclosure. Accordingly, the particular arrangements are illustrative only and are not limiting as to the scope of the invention that is to be given the full breadth of any and all equivalents thereof.

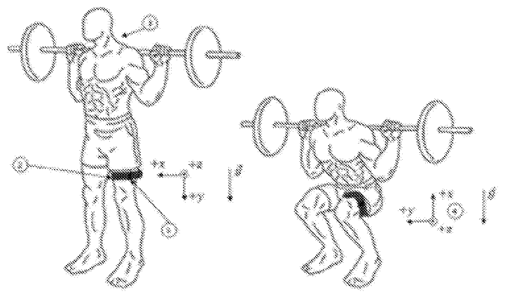

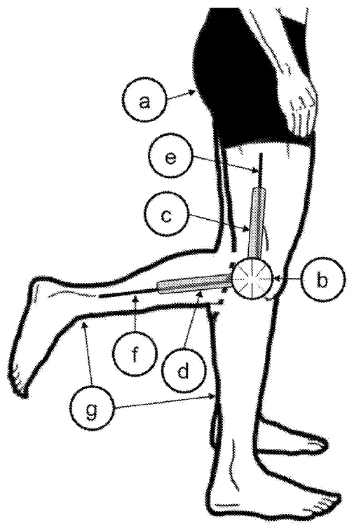

FIG. 9 depicts a knee flexion using a standard goniometer. In FIG. 6, (a) is the user whose joint is being measured for range of motion; (b) shows the center of the goniometer positioned at the center of the knee joint that has tick marks to indicate the angle between the two arms of the goniometer; (c) depicts the first arm of the goniometer that remains stationary from the starting position of the body part (e.g. quadriceps) during the range of motion measurement (d) depicts the second arm of the goniometer that moves along with the body part (e.g. calf) during the range of motion measurement; (e) depicts the femoral shaft where, e.g. the first arm of the goniometer is ideally aligned with; (f) depicts the tibia bone where, e.g., the second arm of the goniometer is ideally aligned with; (g) depicts two arrows pointing at two views of the same leg as it, goes from straight (i.e. approximately 180 degrees) to bent (approximately 90 degrees).

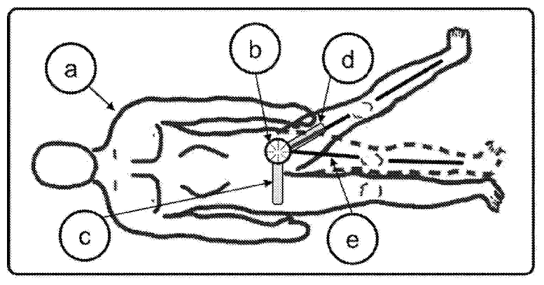

FIG. 10 depicts a hip abduction using a standard goniometer. In FIG. 10, (a) depicts the user whose joint is being measured for range of motion, (b) depicts the center of the goniometer positioned at the center of the hip joint that has tick marks to indicate the angle between the two arms of the goniometer; (c) the first arm of the goniometer remains stationary across the hip and acts as the axis during the range of motion measurement; (d) depicts the second aim of the goniometer that moves along with the body part (e.g. quadriceps) during this range of motion measurement; (e) depicts the femoral shaft which is where the arms of the goniometer are aligned with.

FIG. 11 depicts a hip rotation wherein: (a) points to the person or user; (b) depicts the location of the left hip joint; (c) depicts the rotation around the axis of the leg; and (d) depicts the axis of rotation through the leg.

In addition to range of motion and velocity tracking, our device also provides information on force output, torque and power for specific movements using the values from the accelerometer and quaternions. Because our device relies on motion detection, we cannot measure static force (force applied to an object at rest), only force on an object that is being accelerated. The magnitude of the force applied is given below. In this orientation, the x-y plane is perpendicular to the gravity vector which is oriented along z. In the below equations, in is the mass of the weight (in kilograms) and a is the gravity corrected acceleration values from the spectrometer. It is important to note that for the force along z, the acceleration is added to the gravity vector (9.8 m/s²).

Fₙₑₜ=\sqrt{Fₓ²F_{y}²+F_{z}²}

Fₓ=maₓ

F_{y}=ma_{y}

F_{z}=m(a_{z}+g)

Torque measurements are beneficial for measuring the angular strain on a body joint. A prime example would be the torque on the shoulder joint during a lateral raise or the torque on the knee during a leg extension.

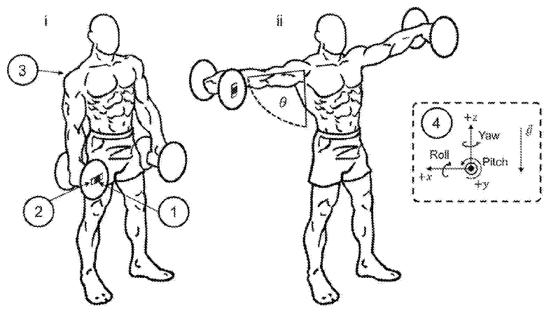

FIG. 12 depicts a side lateral raise wherein: the user is oriented standing holding a dumbbell in each hand with the device attached to the dumbbell, e.g. with a band. The user can perform a shoulder lateral raise movement by raising their arms in a pitch/roll motion according to the coordinate system shown.

In FIG. 12, θ represents the angle between the x-y plane and the z vector while \vec{F}ₚₑᵣₚ is the applied force perpendicular to the lever at a distance d away from the point of rotation. It follows then that the torque is given by:

T=\vec{F}ₚₑᵣₚ·\vec{d}

While we cannot directly measure the applied perpendicular force, we do measure the net force and the angle calculated from the quaternions. The distance between the joint and weight can be easily measured with a tape measure or rule. The torque exerted on a joint is then given by:

T=Fₙₑₜ sin(θ)|\vec{d}|

Additionally, power can be measured using the given values of the device as well as the calculated values provided in this section (velocity, force, etc.). The power of a given movement can be calculated using the following equation:

$$P = \frac{m {v}^{2}}{t}$$

One of the advantages with using this device is that there is no calibration phase i.e., once the device is properly placed on the user, and the user moves, the device will record a change in motion and send the information to the microcontroller for processing.

Another advantage with using this device is that the measurement of the range of motion is independent of the alignment of the device with the bone. So long as the device is placed correctly, it is accurate to less than a degree.

This device measures the relative movement of the joint regardless of the type of joint, and the exact location of the joint, making it much more accurate across a wider range of patient body types.

In addition to the range of motion in tell is of rotation angles, the device can also calculate the acceleration and velocity with which the movement is made And can display that information on the external device.

In some embodiments this device can be used to measure whether specific joint positions as required in sporting events are achieved. In some embodiments, this device can be used for squat depth measurement. In some embodiments, this device can be used for measuring bench presses. In some embodiments, the device can be used for deadlift measurement.

The definition of an acceptable squat in the sport of powerlifting is when the hip joint slightly surpasses the top of the knee resulting in your thigh becoming parallel with the ground. The majority of weight lifters do not practice the sport of powerlifting, but the definition of a squat holds universally true because achieving this minimum “parallel” position results in engaging all of the important muscles that are noted in a squat. The current procedure is that three judges watch a lifter to see if the lifter lifts the weights correctly. Such a manual method of measurement leaves room for errors. Additionally, during practice lifters cannot judge the depths of their squat on their own. This device solves both those issues by measuring the squat depth based on the user's rotation and movement through the coordinate system (3) and the calibration settings in the mobile software and displaying all the information on the mobile application.

To measure the depth of a squat, the device may be attached to the thigh of the user. The IMU then measures the angle and velocity of the user's squat and the microcontroller processes the data to notify the user in real time if his/her squat depth is reached. The microcontroller communicates with the mobile device using the Bluetooth.

A mobile application software on the mobile device or a computer was developed to notify the squatter if depth was achieved, the angle and distance away from depth if not achieved, as well as the velocity and acceleration of each squat repetition, as well as calibration of these settings. The mobile application will store this information within the user profile created by the user.

One example of use for measurement in sports is bench press measurement. The definition of an acceptable bench press in the sport of powerlifting is as follows. The lifter unracks their weight and waits for the press command, which is when the barbell is completely motionless and the lifter is in position 18 in FIG. 8. Once the lifter receives the start command, the lifter brings the barbell down to their chest where the bar must touch their chest and become completely motionless as shown in position 19 in FIG. 8. Once the barbell is touching the lifters chest and the barbell is motionless, the lifter receives the press command. Once the press command is received, the lifter will press the barbell to position 20 in FIG. 8. Once the lifter is motionless in position 20, the lifter receives the rack command where the lifter proceeds to place their weight back in the rack.

To measure the bench press, the device is attached to the barbell. The IMU of the device measures the velocity and angle of the user's movement to using the coordinate system (15) in FIG. 8 and sends the data to the microcontroller. The microcontroller then processes with respect to gravity and the rotation of the barbell (16) and (17) in FIG. 8 and sends the information to the mobile device to be displayed.

The device communicates with the mobile application to let the user know in real time if the barbell has any pitch or yaw and if the start, press and rack commands are achieved Mobile application software on the mobile device or a computer was developed to notify the user if the start, press and rack commands are achieved, the pitch and yaw of the barbell, the velocity and acceleration of each bench press repetition, as well as calibration of these settings. The mobile application will store this information within the user profile created by the user.

Medicine Ball Tracking

Explosive exercises such as weight lifting (e.g., clean-and-jerk and barbell snatch) and barbell squat jumps which are used as training methods that simulate real sporting movements for explosiveness are limited in the real sporting movement practicality. The limitation of these movements is the fact that the body naturally decelerates itself when performing these exercises because the barbell does not leave the hand. Since the barbell does not leave the hand during these exercises, it is impossible to train for peak power output. A solution to this problem is training with medicine balls. Athletes throw medicine balls as a way to develop the ultimate measure of peak power output because the ball is being released from the user's hand, which allows the user to achieve their peak power output the medicine balls are fairly light compared to the traditional explosive weight lifting exercises which allows for more accurate sports movements similar to that of real competition and this inherently allows the athlete to perform the exercises as fast as possible.

In addition to range of motion, general velocity, acceleration, force, torque and power measurements, our device also can be used to measure velocity, acceleration, force, power and torque of medicine ball exercises. Our device is placed in a protective enclosure around the spherical medicine ball. Upon performing an exercise involving throwing a medicine ball, the user will prompt the app to tell the device to begin measuring the movement either on screen or voice command. Upon completing the exercise, the user will prompt tell the device to stop

What is claimed is:

1. A range of motion measuring device, comprising:
a gyroscope;
an accelerometer;
a magnetometer; and
an external device to display the results;
wherein the device uses a mathematical formula to determine a range of motion of a user using a set of data received.

2. The device of claim 1, wherein the device has a microcontroller.

3. (canceled)

4. The device of claim 1, wherein the microcontroller and internal measurement unit communicate wirelessly.

5. The device of claim 1, wherein the microcontroller processes the information sent by the internal measurement unit.

6. The device of claim 1, wherein the microcontroller sends the processed information to the external device for display.

7. The device of claim 1, wherein a battery is rechargeable.

8. (canceled)

9. The device of claim 1, wherein a USB port is used to charge a battery.

10. The device of claim 1, wherein the USB port is used to calibrate the microcontroller.

11. (canceled)

12. The device of claim 1, wherein the external device is capable of computing and displaying results.

13. (canceled)

14. (canceled)

15. A device to measure athletic movements, comprising: a gyroscope;
an accelerometer;
a magnetometer; and
an external device to display the results;
wherein the device uses quaternions to calculate a user's range of motion.

16. The device of claim 15, wherein the device has a microcontroller.

17. (canceled)

18. The device of claim 15, wherein the microcontroller and internal measurement unit communicate wirelessly.

19. The device of claim 15, wherein the microcontroller processes the information sent by the internal measurement unit.

20. The device of claim 15, wherein the microcontroller sends the processed information to the external device for display.

21. (canceled)

22. The device of claim 15, wherein a battery is non-rechargeable.

23. The device of claim 15, wherein a USB port is used to charge a battery.

24. The device of claim 15, wherein the USB port is used to calibrate the microcontroller.

25. (canceled)

26. The device of claim 15, wherein the external device is capable of computing and displaying results.

27. (canceled)

28. The device of claim 15, wherein the encased device is temporarily attached to a body of the subject with a band.

29. The device of claim 15 wherein the device is attached to a medicine ball.

30. (canceled)
